(19) United States
(12) Patent Application Publication
(10) Pub. No.: US 2019/0231788 A1
(43) Pub. Date: Aug. 1, 2019

(54) COMPOSITIONS COMPRISING TIMOLOL AND AN ANTI-INFLAMMATORY AGENT

(71) Applicant: Almirall, S.A., Barcelona (ES)

(72) Inventors: Nuria Godessart Marina, Barcelona (ES); Gema Tarrason Encuentra, Barcelona (ES)

(73) Assignee: Almirall, S.A., Barcelona (ES)

(21) Appl. No.: 16/309,814

(22) PCT Filed: Jun. 15, 2017
(86) PCT No.: PCT/EP2017/064707

§ 371 Date: Dec. 13, 2018

(30) Foreign Application Priority Data
Jun. 16, 2016 (EP) 16382279.4

(51) Int. Cl. A61K 31/5377; A61K 31/7048; A61K 31/4164; A61K 31/4985; A61K 9/00; A61K 9/06; A61K 47/44; A61P 17/00

(52) U.S. Cl. CPC A61K 31/5377; A61K 31/7048; A61K 31/4164; A61P 17/00; A61K 9/0014; A61K 9/06; A61K 47/44; A61K 31/4985

(57) ABSTRACT

The present invention relates to combinations comprising timolol and a compound useful for the treatment of rosacea selected from ivermectin, metronidazole and praziquantel and to the use of these combinations in the topical treatment of rosacea.

The present invention relates to combinations comprising timolol and a compound useful for the treatment of rosacea selected from ivermectin, metronidazole and praziquantel and to the use of these combinations in the topical treatment of rosacea.

### BACKGROUND TO THE INVENTION

Rosacea is a common chronic-recurrent, usually symmetrical facial, dermatosis that persists for years with periods of exacerbation and remission. It is a chronic inflammatory cutaneous disease primarily affecting the central face of adults aged between 25 and 70. Rosacea can be categorized into four subtypes: (1) erythematotelangiectatic rosacea (ETR) defined by the presence of flushing and central facial erythema, (2) papulopustular rosacea (PPR) defined by the presence of persistent erythema and transient papules or pustules, (3) phymatous rosacea, presenting with thick skin, irregular surface nodularities, and enlargement of face skin surfaces such as the nose (rhinophyma), and (4) an ocular subtype that presents as dryness, irritation, blepharitis, conjunctivitis, or keratitis, and that can compromise eyesight.

Rosacea occurs both in men and women, although there are some gender differences. It usually starts earlier among females, whereas rhinophyma is almost exclusively seen among males. Rosacea is most frequently observed in patients with fair skin and conservative estimates suggest that the disease affects 14 million individuals in the US alone, at a prevalence of 5%. It has an impact on patients' quality of life, since their physical appearance negatively influences their social and emotional health.

Conventional treatments for rosacea have focused on the inflammatory lesions, pustules and papules. Typically anti-microbial metronidazole, azelaic acid or sodium sulfacetamide-sulphur topical formulations are used to treat subtype 2 (PPR). Oral tetracyclines, such as doxycyline and minocycline, are also widely used for systemic treatment in rosacea subtypes 2 and 4. Recently, modified release formulations of low dose doxycycline and minocycline have been developed to minimize gastrointestinal side effects and concern about antibiotic resistance. Also recently, topical ivermectin, an anti-helmintic drug, has been approved for the treatment of inflammatory lesions of rosacea.

Brimonidine tartrate, an agonist of the a2 adrenergic receptors, in a gel formulation at 0.5% has been recently approved for the treatment of nontransient facial erythema acting on the cutaneous vascular component of the disease. However, brimonidine has been reported to induce transient worsening of the erythema and flushing in some patients, raising some concerns about its utility.

In spite of this varied range of oral and topical compounds, proper control of the disease has not yet been achieved, in particular of the most common clinical subtypes 1 and 2.

Therefore, there have been attempts to combine compounds having different mechanisms of action in order to increase the overall efficacy of the treatment. However, due to the difficulties posed by the characteristics of the skin to the effectiveness of topical drugs, there are no topical combination treatments for rosacea available in the market.

It has now surprisingly been found that topical combinations of timolol with other active compounds useful for the treatment of rosacea have a significant increase in efficacy, for example in their ant inflammatory activity, compared with the effects of each component of the combination when used as a monotherapy. Thus, the combinations of the invention would be particularly advantageous for treating both the facial erythema and the inflammatory lesions and papules which characterises rosacea. The use of timolol has the additional advantage over alpha-1 or alpha-2 adrenergic receptor agonists, like brimonidine, that it is devoid of a rebound erythema effect. In addition, the topical administration of the combinations defined above avoids the side effects which would result from oral administration.

### SUMMARY OF THE INVENTION

The present invention therefore provides a combination for topical use which comprises (a) timolol or a pharmaceutically acceptable salt thereof, and (b) an active compound useful for the treatment of rosacea selected from ivermectin, metronidazole, praziquantel, and pharmaceutically acceptable salts thereof.

The present invention also provides a combination as defined above, for use in treating rosacea.

The present invention further provides a pharmaceutical composition suitable for topical administration comprising a combination as defined above. In a preferred embodiment, components (a) and (b) are present in a single formulation.

The present invention also provides timolol or a pharmaceutically acceptable salt thereof for use in treating rosacea, by topical co-administration with an active compound as defined above.

The present invention also provides an active compound as defined above for use in treating rosacea, by topical co-administration with timolol or a pharmaceutically acceptable salt thereof.

The present invention also provides a method of treating a patient suffering from rosacea, which method comprises topically administering to said patient a combination or a pharmaceutical composition as defined above.

The present invention further provides a method of treating a patient suffering from rosacea, which method comprises topically co-administering to said patient (a) timolol or a pharmaceutically acceptable salt thereof and (b) an active compound as defined above.

The present invention also provides the use of a combination or of a pharmaceutical composition as defined above for the manufacture of a medicament for the topical treatment of rosacea.

The present invention further provides the use of (a) timolol or a pharmaceutically acceptable salt thereof in the manufacture of a medicament for the topical treatment of rosacea by co-administration with (b) an active compound as defined above.

The present invention further provides the use of (b) an active compound as defined above in the manufacture of a medicament for the topical treatment of rosacea by co-administration with (a) timolol or a pharmaceutically acceptable salt thereof.

In a particularly preferred embodiment of the invention timolol is in the form of timolol maleate.

In the present invention co-administration and co-administering means that components (a) and (b) of the combination are topically administered to the patient in a simultaneous, concurrent, concomitant, separate or sequential way.

### BRIEF DESCRIPTION OF THE FIGURES

FIG. 1 is a comparison of the anti-oedema effect of timolol, ivermectin and a combination thereof after a single topical application in the TPA-induced mouse ear oedema model.

FIG. 2 is a comparison of the anti-oedema effect of timolol, praziquantel and a combination thereof after a single topical application in the TPA-induced mouse ear oedema model.

FIGS. A, B and C are the anti-oedema dose-responses of ivermectin, praziquantel and timolol after a single topical application in the TPA-induced mouse ear oedema model, respectively, bases for the dose selection of each compound for the combination studies.

### DETAILED DESCRIPTION OF THE INVENTION

Timolol is (S)-1-(tert-butylamino)-3-[(4-morpholin-4-yl-1,2,5-thiadiazol-3-yl)oxy]propan-2-ol. It has the structure:

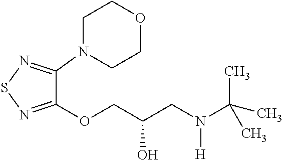 [SMILES: CC(C)(C)NC[C@H](O)COc1nsnc1N1CCOCC1]

Ivermectin can exist as a pair of homologue compounds, with a major component (B₁ₐ) and a minor component (B_{1b}). Ivermectin B₁ₐ is 22,23-dihydroavermectin B₁ₐ and Ivermectin B_{1b} is 22,23-dihydroavermectin B_{1b}. They differ only in the presence of an extra methylene group in ivermectin B₁ₐ and their structures are as follows:

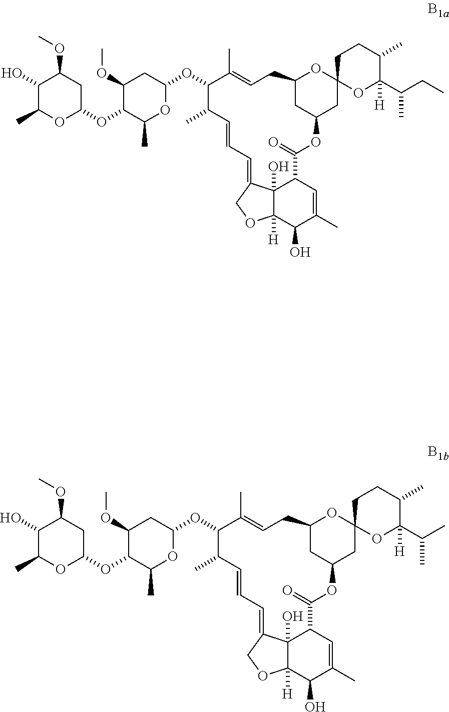

As used herein, references to “ivermectin” are intended to embrace compound B₁ₐ, compound B_{1b}, and mixtures thereof. Preferably, the ivermectin used in the present invention is a mixture of B₁ₐ and B_{1b}. More preferably it is a mixture having a major component of B₁ₐ and a minor component of B_{1b}.

Metronidazole is 2-(2-methyl-5-nitro-1H-imidazol-1-yl)ethanol and has the following structure:

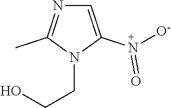

Praziquantel is (RS)-2-(Cyclohexylcarbonyl)-1,2,3,6,7,11b-hexahydro-4H-pyrazino[2,1-a]isoquinolin-4-one and has the following structure:

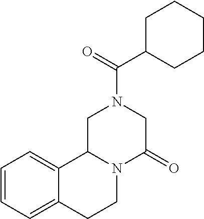 [SMILES: O=C(C1CCCCC1)N1CC(=O)N2CCc3ccccc3C2C1]

The present invention provides pharmaceutical compositions comprising timolol or a pharmaceutical acceptable salt thereof and an active compound useful for the treatment of rosacea selected from ivermectin, metronidazole, praziquantel, and pharmaceutically acceptable salts thereof for use in treating rosacea. Standard principles underlying the selection and preparation of pharmaceutically acceptable salts are described, for example, in Handbook of Pharmaceutical Salts: Properties, Selection and Use, ed. P. H. Stahl & C. G. Wermuth, Wiley-VCH, 2002. Suitable pharmaceutically acceptable salts of the compounds for use in this invention include addition salts with a pharmaceutically acceptable acid such as such as hydrochloric acid, sulphuric acid, methanesulphonic acid, fumaric acid, maleic acid, succinic acid, acetic acid, benzoic acid, citric acid, tartaric acid or phosphoric acid. Other salts may be formed with a pharmaceutically acceptable base. Suitable such pharmaceutically acceptable salts include alkali metal salts, e.g. sodium or potassium salts; alkaline earth metal salts, e.g. calcium or magnesium salts; ammonium salts; and salts formed with suitable organic ligands, e.g. quaternary ammonium salts, and meglumine salts.

In a preferred embodiment of the invention the timolol compound is timolol. In a further preferred embodiment of the invention the timolol compound is timolol maleate.

In a preferred embodiment of the invention the active compound (b) of the composition is ivermectin or a pharmaceutically acceptable salt thereof.

In a preferred embodiment of the invention the active compound (b) of the composition is praziquantel or a pharmaceutically acceptable salt thereof.

In a preferred embodiment of the invention the active compound (b) of the composition is metronidazole or a pharmaceutically acceptable salt thereof.

Pharmaceutical compositions according to the invention will typically further comprise one or more pharmaceutically acceptable excipients or carriers.

Typically the rosacea to be treated is erythematotelangiectatic rosacea or papulopustular rosacea. In another embodiment the rosacea to be treated is papulopostular rosacea, phymatous rosacea or rosacea subtype 4 (ocular rosacea). The combination therapy of the present invention is particularly useful in treating erythema and oedema caused by rosacea.

The combination therapy of the present invention it is also useful in treating the inflammatory lesions and papules described in papulopustular rosacea.

Typically the patient to be treated is a mammal. Preferably the patient is a human. More preferably the patient is a Caucasian human.

In a preferred embodiment of the invention both active ingredients (a) and (b) are present in the same topical pharmaceutical composition or formulation. However, the present invention extends to situations where the active ingredients discussed above are co-administered in separate pharmaceutical compositions which are suitable for topical administration.

Co-administration of the active ingredients according to the present invention includes simultaneous, separate and sequential administration. Typically, both drugs are administered simultaneously or one drug is administered first and the second drug is administered within 12 hours, preferably within 6 hours, more preferably within 3 hours, most preferably within 1 hour after the administration of the first drug.

Typically, the active ingredients discussed above, the pharmaceutically acceptable salts thereof or the pharmaceutical compositions of the present invention are applied topically to the face of a patient. Typically they are not applied around the eyes. More typically, they are not applied within 0.2 cm, more typically not within 0.5 cm, preferably not within 1 cm, of the eye.

Typically, the active ingredients discussed above, the pharmaceutically acceptable salts thereof or the pharmaceutical compositions of the present invention are for use other than in conjunction with laser treatment, in particular Intense Pulsed Light (IPL) laser treatment. Thus, typically the patient treated according to the invention is not undergoing, and preferably has not been subjected to, such laser treatment.

For topical administration the pharmaceutical compositions of the present invention may take the form of any formulation normally used for topical administration, in particular solutions, lotions, emulsions of liquid consistency, emulsions of semi-liquid consistency, emulsions of semi-solid consistency, emulsions of solid consistency, creams, gels or ointments. In a particular embodiment, the pharmaceutical composition of the present invention is a lotion, a cream or a gel; preferably a lotion or a cream.

The emulsions are obtained by dispersion of an oil phase in water (O/W) or a water phase in oil (W/O). Preferred pharmaceutical compositions for topical administration contain an oil phase. Preferably, said pharmaceutical compositions are water-in-oil emulsions (i.e. emulsions wherein the water is the dispersed phase and the oil in the dispersion medium) or oil-in-water emulsions (i.e. emulsions wherein the oil is the dispersed phase and the water in the dispersion medium).

Compositions for topical use in accordance with the invention may also contain one or more emollients, emulsifiers, thickeners and/or preservatives.

The emollients are typically long chain alcohols, such as cetyl alcohol, stearyl alcohol and cetearyl alcohol; hydrocarbons such as petrolatum and light mineral oil; or acetylated lanolin. The total amount of emollient in the formulation is preferably about 5% to about 30%, and more preferably about 5% to about 10% by weight based on the total weight of the formulation.

The emulsifier is typically a nonionic surface active agent, e.g., polysorbate 60 (available from Sigma Aldrich), sorbitan monostearate, polyglyceryl-4 oleate, and polyoxyethylene(4)lauryl ether or trivalent cationic. Generally the total amount of emulsifier is preferably about 2% to about 14%, and more preferably about 2% to about 6% by weight based on the total weight of the formulation.

Pharmaceutically acceptable thickeners, such as Veegum™ K (available from R. T. Vanderbilt Company, Inc.), and long chain alcohols (i.e. cetyl alcohol, stearyl alcohol or cetearyl alcohol) can be used. The total amount of thickener present is preferably about 3% to about 12% by weight based on the total weight of the formulation.

Preservatives such as methylparaben, propylparaben and benzyl alcohol can be present in the formulation.

Optionally, an additional solubilizing agent such as benzyl alcohol, lactic acid, acetic acid, stearic acid or hydrochloric acid can be included in the formulation.

Optionally, the formulation can contain a humectant such as glycerin and skin penetration enhancers such as butyl stearate.

It is known to those skilled in the art that a single ingredient can perform more than one function in a composition, i.e., cetyl alcohol can serve both as an emollient and as a thickener.

Preferably, the pharmaceutical composition of the invention comprises an oil phase.

Typically, the amount of oil in the composition is at least 10 wt. %, preferably at least 15 wt. %, more preferably at least 20 wt. %, still more preferably at least 25 wt. %, based on the total weight of the composition. As used herein an oil phase is typically a liquid or solid phase which is substantially immiscible with water. More typically, an oil phase as used herein has a solubility in water at 25° C. of less than or equal to 1 mg/L, preferably less than 0.1 mg/L.

The oil phase in an emulsion may be any oil phase normally used in emulsions for topical administration. Such oil phases include, for example, hydrocarbon bases such as such as hard paraffin, soft paraffin, ceresine and microcrystalline wax, absorption bases such as lanolin and beeswax, emulsifying bases such as emulsifying wax and cetrimide, and vegetable oils such as olive oil, coconut oil, sesame oil, almond oil and peanut oil. Other oil phases useful in accordance with the invention are mineral oil, liquid petroleum, sorbitan monostearate, polysorbate 60, cetyl esters wax, cetearyl alcohol, benzyl alcohol and 2-octyldodecanol.

Those skilled in the art will understand that by varying the ratio of water to oil in an emulsion, the result could be deemed a lotion, a cream, or an ointment, by order of increasing proportion of oil. An emulsion comprising similar proportions of oil phase and water phase is usually deemed a cream, whereas an ointment will generally contain a substantially higher proportion of oil phase compared to water phase, for example greater than 60 wt. % oil phase, preferably greater than 70 wt. % oil phase, more preferably greater than 80 wt. % oil phase, based on the total weight of the oil phase and the water phase. A lotion will generally contain a lower proportion of oil phase than a cream, for example under 25 wt. % oil phase, under 20 wt. % oil phase, under 15 wt. % oil phase, under 10 wt. % oil phase or under 5 wt. % oil phase, based on the total weight of the oil phase and the water phase.

Generally, a cream for use according to the invention comprises an oil phase and a water phase mixed together to form an emulsion. Preferably, the amount of water present in a cream of the invention is about 15% to about 70% by weight based on the total weight of the cream, more preferably about 20 wt. % to about 60 wt. %, even more preferably about 30 wt. % to about 55 wt. %.

Where the composition is an ointment a pharmaceutically acceptable ointment base will be used. Examples of ointment bases include hydrocarbon bases such as such as hard paraffin, soft paraffin, ceresine and microcrystalline wax, absorption bases such as lanolin and beeswax, water-soluble bases such as polyethylene glycols (e.g. polyethylene glycol 200, 300, 400, 3350, 4000 or 6000), propylene glycol and polypropylene glycols, emulsifying bases such as emulsifying wax and cetrimide, and vegetable oils such as olive oil, coconut oil, sesame oil, almond oil and peanut oil. Mixtures of ointment bases can of course be used. The amount of ointment base present in an ointment of the invention is preferably about 60% to about 95% by weight based on the total weight of ointment, more preferably about 70 wt. % to about 90 wt. %, still more preferably about 75 wt. % to about 85 wt. %.

The pharmaceutical composition for use in accordance with the present invention may also be a lotion containing the active component suspended or dissolved in one or more pharmaceutically acceptable carriers. Particular carriers include, for example, mineral oil, sorbitan monostearate, polysorbate 60, cetyl esters wax, cetearyl alcohol, benzyl alcohol, 2-octyldodecanol and water.

Typically, the pharmaceutical composition of the invention contains less than 70 wt. % water, preferably less than 60 wt. % water, based on the total weight of the composition.

Pharmaceutical compositions for use according to the present invention may be substantially non-aqueous. Typically, a substantially non-aqueous pharmaceutical composition comprises less than 25% water by weight, relative to the total weight of the composition, preferably less than 20%, more preferably less than 15%, even more preferably less than 10%, more preferably still less than 5%, still more preferably less than 2% and most preferably less than 1% water.

In the compositions according to the invention, the timolol or pharmaceutically acceptable salt thereof and anti-inflammatory agent selected from ivermectin, metronidazole and praziquantel and pharmaceutical salts thereof, may each be present at a concentration of between 0.001 and 20% by weight, relative to the total weight of the composition, preferably between 0.01 and 10%, more preferably between 0.1 and 5% by weight, in particular 0.1%, 0.25%, 0.3%, 0.35, 0.4%, 0.45%, 0.5%, 0.55%, 0.6%, 0.65%, 0.7%, 0.75%, 1%, 1.25%, 1.5%, 1.75%, 2%, 2.25%, 2.5%, 2.75%, 3%, 3.25%, 3.5%, 3.75%, 4%, 4.25%, 4.5%, 4.75% or 5%.

In a particular embodiment, timolol is present at 1% by weight and ivermectin at 1% by weight. In another particular embodiment, timolol is present at 2% by weight and ivermectin at 1% by weight. In another particular embodiment, timolol is present at 3% by weight and ivermectin at 1% by weight. In a particular embodiment, timolol is present at 0.1% by weight and ivermectin at 1% by weight. In another particular embodiment, timolol is present at 0.5% by weight and ivermectin at 1% by weight.

In a particular embodiment, timolol is present at 1% by weight and praziquantel is present at 1% by weight. In another particular embodiment, timolol is present at 2% by weight and praziquantel is present at 1% by weight. In another particular embodiment, timolol is present at 3% by weight and praziquantel is present at 1% by weight.

In a particular embodiment, timolol is present at 1% by weight and praziquantel is present at 2% by weight. In another particular embodiment, timolol is present at 2% by weight and praziquantel is present at 2% by weight. In another particular embodiment, timolol is present at 3% by weight and praziquantel is present at 2% by weight.

In a particular embodiment, timolol is present at 1% by weight and praziquantel is present at 3% by weight. In another particular embodiment, timolol is present at 2% by weight and praziquantel is present at 3% by weight. In another particular embodiment, timolol is present at 3% by weight and praziquantel is present at 3% by weight.

In a particular embodiment, timolol is present at 1% by weight and metronidazole at 1% by weight. In another particular embodiment, timolol is present at 2% by weight and metronidazole at 1% by weight. In another particular embodiment, timolol is present at 3% by weight and metronidazole at 1% by weight.

In another particular embodiment, timolol is present at 1% by weight and metronidazole at 0.75% by weight. In another particular embodiment, timolol is present at 2% by weight and metronidazole at 0.75% by weight. In another particular embodiment, timolol is present at 3% by weight and metronidazole at 0.75% by weight.

In all the above particular embodiments, the percentage of timolol is expressed as timolol free base.

The present invention is explained in more detail in the following by referring to Example below, which is not to be construed as limitative.

### EXAMPLE

Evaluation of the Anti-Oedema Effect of Ivermectin, Praziquantel and Timolol and Combinations of Timolol with Ivermectin or Praziquantel after a Single Topical Application in the TPA-Induced Ear Oedema Model in Balb/c Mice.

D. Piwnica et al. described in J. Dermatol. Sci., 75 (1) 49-54, 2014, that topical brimonidine 0.2% inhibited by 50% TPA-induced ear oedema in mice. According to these authors, this confirms that the “reduction of oedema in rosacea is a critical requirement for any new treatment”.

In view of these findings, the inventors of the present application used the same model to check whether the combinations of timolol of the invention would be more beneficial in the treatment of rosacea than the compounds administered as a monotherapy.

Experimental Design:

The oedema is induced by means of a single application to the right ear of the mouse of 10 μl of a solution of TPA (phorbol 12-myristate 13-acetate) in acetone at 0.01%. The test compounds are diluted in acetone and applied with TPA. The weight of the mouse ears is measured at T+6 h and the weight of the left ear is subtracted from the one of the right ear.

Treatments:
A. Ivermectin is applied at a concentration of 0.1-0.3-1%,
B. Praziquantel is applied at a concentration at 0.3-1-3%
C. Timolol is applied at a concentration at 0.2-0.5-1%.

For combination studies Ivermectin alone is applied at a concentration of 0.3%, praziquantel alone is applied at 1% and timolol alone is applied at 0.5%. These same strengths are used in the combinations.

Results:

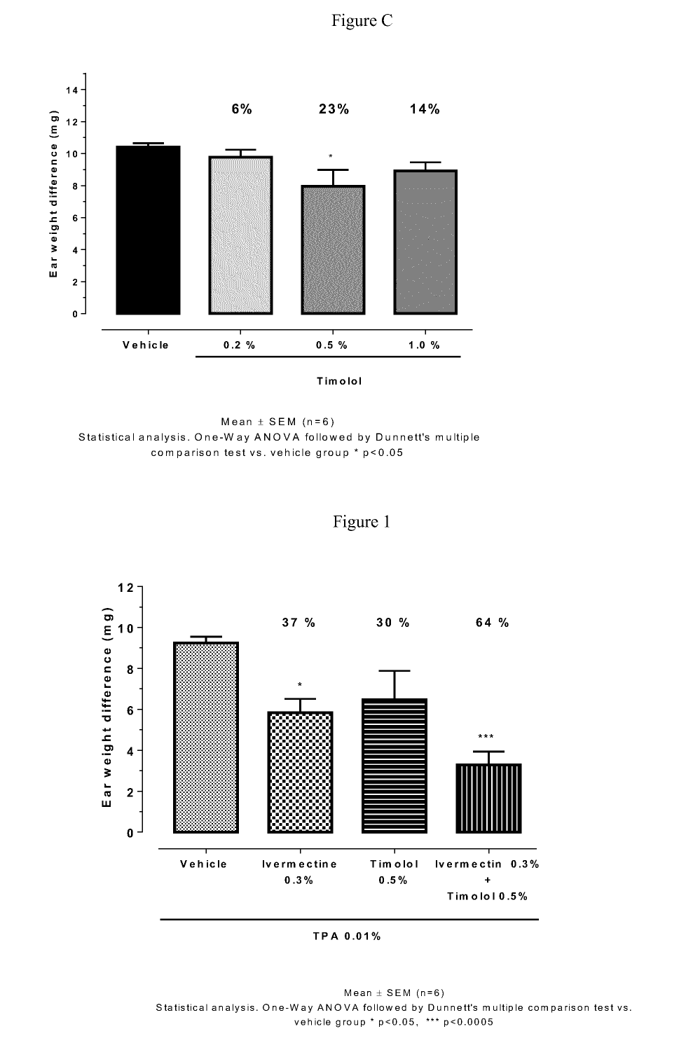

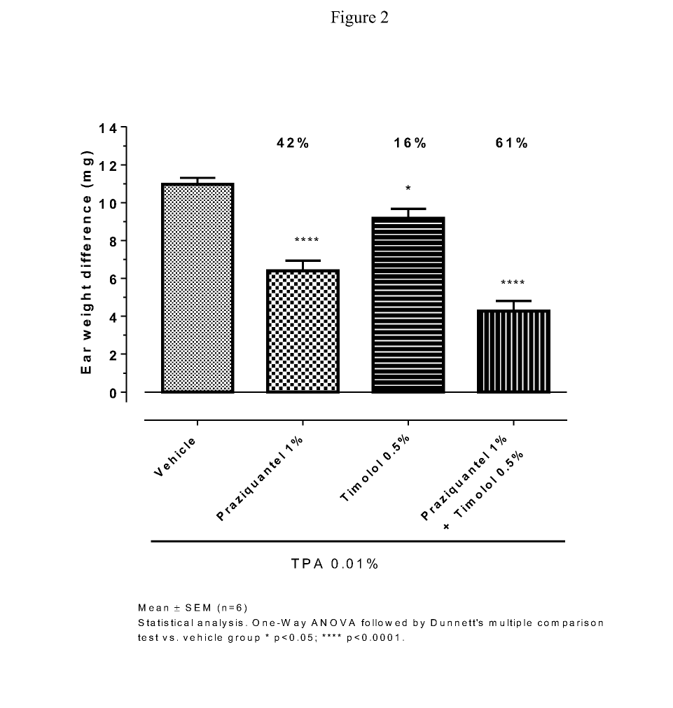

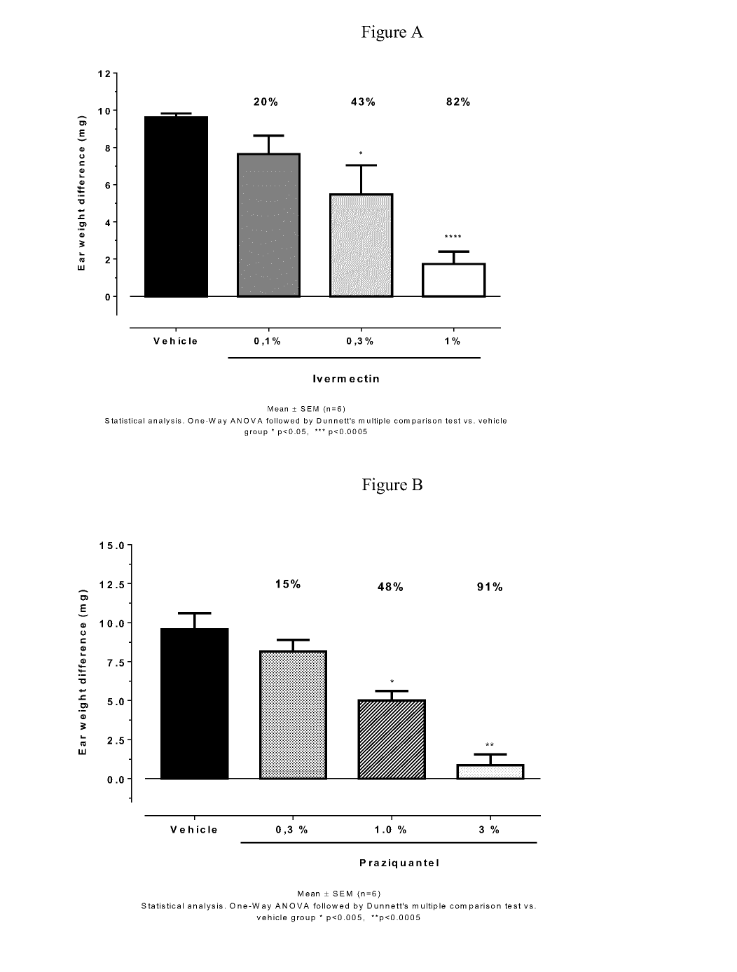

The results of the experiments are shown in FIGS. A, B and C and FIGS. 1 and 2. Each graph represents the average weight of the ear oedema of four different groups of treated animals. Each group includes 6 animals. Numbers above the bars indicate the percentage inhibition of the corresponding group of treated animals vs the control group.

Results in FIG. A show that ivermectin at 0.1-0.3-1% was able to inhibit ear oedema by 20, 43 and 82% respectively

Results in FIG. B show that praziquantel 0.3-1-3% was able to inhibit ear oedema by 15, 48 and 91% respectively.

Results in FIG. C show that timolol at 0.2-0.5-1% slightly inhibited ear oedema by 6, 23 and 14% respectively.

These dose-response studies served as basis to define submaximal doses for the combination studies; ivermectin at 0.3%, and praziquantel at 1% were the doses selected to be combined with 0.5% timolol.

In FIG. 1 Ivermectin at 0.3% inhibited ear oedema by 37%, timolol 0.5% by 30% and the combination of both drugs inhibited ear oedema formation by 64%. In FIG. 2 Praziquantel at 1% inhibited ear oedema by 42%, timolol 0.5% by 16% and the combination of both drugs inhibited TPA induced oedema by 61%.

These results show that the combined administration of timolol with ivermectin or praziquantel produces a significant anti-oedema effect, which is greater than the effect of each of the compounds administered alone. The combinations of timolol of the invention would be thus particularly beneficial in the treatment of rosacea.

1. A combination for topical use which comprises (a) timolol or a pharmaceutically acceptable salt thereof, and (b) an active compound useful for the treatment of rosacea selected from ivermectin, metronidazole, praziquantel, and pharmaceutically acceptable salts thereof.

2. The combination according to claim 1, wherein component (a) of the combination is timolol or timolol maleate.

3. The combination according to claim 1, wherein component (b) of the composition is ivermectin or a pharmaceutically acceptable salt thereof.

4. The combination according to claim 1, wherein component (b) of the composition is praziquantel or a pharmaceutically acceptable salt thereof.

5. The combination according to claim 1, wherein component (b) of the composition is metronidazole or a pharmaceutically acceptable salt thereof.

6. (canceled)

7. A pharmaceutical composition suitable for topical administration comprising (a) timolol or a pharmaceutically acceptable salt thereof, and (b) an active compound useful for the treatment of rosacea selected from ivermectin, metronidazole, praziquantel, and pharmaceutically acceptable salts thereof, wherein the components (a) and (b) are present in the same formulation.

8. The pharmaceutical composition according to claim 7, wherein the composition comprises an oil phase.

9. The pharmaceutical composition according to claim 7, wherein the composition is a lotion, a cream or a gel.

10. The pharmaceutical composition according to claim 7 which contains less than 60 wt. % water, based on the total weight of the composition.

11-13. (canceled)

14. A method of treating a patient suffering from rosacea which method comprises:
topically administering to said patient a combination for topical use comprising (a) timolol or a pharmaceutically acceptable salt thereof, and (b) an active compound useful for the treatment of rosacea selected from ivermectin, metronidazole, praziquantel, and pharmaceutically acceptable salts thereof; or
co-administering to said patient (a) timolol or a pharmaceutically acceptable salt thereof and (b) an active compound useful for the treatment of rosacea selected from ivermectin, metronidazole, praziquantel, and pharmaceutically acceptable salts thereof; or
topically administering to said patient a pharmaceutical composition suitable for topical administration comprising (a) timolol or a pharmaceutically acceptable salt thereof, and (b) an active compound useful for the treatment of rosacea selected from ivermectin, metronidazole, praziquantel, and pharmaceutically acceptable salts thereof, wherein the components (a) and (b) are present in the same formulation.

15-18. (canceled)
